(19) United States
(12) Patent Application Publication
(10) Pub. No.: US 2005/0239039 A1
(43) Pub. Date: Oct. 27, 2005

(54) Vital mammalian heart tissue cells maintained ex vivo, processes of the collection and cultivation thereof and their use

(76) Inventors: Andreas Goette, Magdeburg (DE); Peter Rohnert, Magdeburg (DE); Uwe Lendeckel, Magdeburg (DE); Frank Striggow, Gerwisch (DE)

Correspondence Address: HODGSON RUSS LLP, ONE M & T PLAZA, SUITE 2000, BUFFALO, NY, 14203-2391, US

(21) Appl. No.: 10/851,834

(22) Filed: May 21, 2004

(30) Foreign Application Priority Data
May 21, 2003 (DE) 10322986.8

(51) Int. Cl. A01N 1/02; C12N 5/08

(52) U.S. Cl. 435/1.1; 435/366

(57) ABSTRACT

The present invention relates to vital mammalian heart tissue cells maintained ex vivo while simulating physiological conditions, particularly vital mammalian heart tissue cells maintained ex vivo in a usual culture medium with the addition of a culture gas at a physiologically acceptable pH value, processes for their collection and for their cultivation as well as their use in model investigations of chemical, biological and/or physical influences on physiological or pathophysiological processes.

The invention relates to vital mammalian heart tissue cells maintained ex vivo, to processes for the collection and cultivation thereof and to their use. In particular, the invention relates to vital human or animal heart tissue cells which are maintained ex vivo, to a process for collecting and cultivating functionally intact human or animal heart tissue as well as to its use for experiments relating to the influence of different chemical, biological and physical parameters as, for example, electric stimulation, ischemia, hypoxia, molecular biological manipulation, application of biologically active substances, on physiological and pathophysiological processes. Thereby, more particularly, changes in an intact tissue unit may be examined and determined, in addition to sub-cellular and cellular changes.

Heart muscle tissue predominantly consists of a dense unit of myocytes, fibroblasts and endothelial cells. Several investigations revealed that the interaction, between each other, of cells and cell populations has great importance for the molecular regulation of various signaling processes. For example, the effect mediated by angiotensin II may be based on a paracrine action. Furthermore, the effect of various pharmacological substances as, for example, antiarrhythmic agents, seems to be influenced by the composition of the myocardial tissue unit. This may be one reason why the examination of the whole and intact tissue is of specific importance for the evaluation of biological and molecular processes and may represent a new quality of the investigations. Up to now, such investigations were possible only with great interventional efforts and with considerable risks of repetitive myocardial biopsies from the patients' ventricles or in whole animal experiments, respectively. Repetitive biopsies of the atrial wall are not possible, neither in vivo in patients nor in animal experiments, since the biopsy almost always results into a perforation of the heart wall. Hence, such biopsies have to be particularly excluded as a regular measure for collecting, for example, human heart atrial tissue. Thus, functional experiments with such tissue were completely impossible up to now.

Hence, the invention had as an object to provide processes for collecting and cultivating mammalian heart tissue cells and—as a result—to also provide mammalian heart tissue cells themselves. In this connection, the cells collected and cultivated may be maintained ex vivo and, nevertheless, are functionally intact. Furthermore, the invention had the object to indicate uses for such vital mammalian heart tissue cells maintained ex vivo.

The invention is based on the surprising effect that a myocardial tissue cut or tissue block, respectively, which was taken from a mammal (mammalia), preferably which was taken from a human, may be maintained in the form of a vital, functionally intact tissue unit for several days ex vivo in a culture. As a result, the present invention allows for the first time functional experiments with heart tissue, in particular with human atrial tissue, to be carried out.

Particularly surprising and unexpected was the fact that changes of the atrial expression pattern which previously could be demonstrated in patients with chronical atrial fibrillation occurred after an electrical stimulation of the tissue cuts collected and cultivated in accordance with the invention (simulation of atrial fibrillation). Moreover, there could be demonstrated surprisingly an induction of the mRNA expression of the κ-opiate receptor at mammalian heart tissue cuts after an electrical stimulation thereof, which expression can be observed in another established model, i. e. in cardiomyocytes (P 19 cells) which were differentiated in vitro from precursor cells, in an equal manner after an electric stimulation thereof.

Hence, the present invention allows the effects of various chemical substances (pharmaceutical substances as, for example, antiarrhythmic drugs, receptor inhibitors, natural and synthetic peptides etc.) or, respectively, the effects of various environmental conditions (various culture media, deprivation of oxygen, hypoglycemia, hyperglycemia, changes of the concentration of electrolyte(s), electrical current etc.) on the myocardial tissue culture to be examined, which was obtained in accordance with the invention. In addition, the model allows tests for the regeneration of changes of the myocardial tissue to be carried out, which tissue changes were induced by external influences.

Furthermore, the invention allows surprisingly—in contrast to the cell cultures from isolated myocytes employed up to now—the examination of the intact tissue unit to be carried out. The functional analysis of the interaction of various cells, in a cell unit at an electro-physiological level and at a molecular level as well, cannot be examined with culture systems consisting of myocytes and fibroblasts employed up to now, the less so since fetal cells are often used for a cultivation of myocytes, which fetal cells are comparable to adult human myocytes in their properties under specific conditions only.

Hence, in a first aspect, the invention relates to vital mammalian heart tissue cells maintained ex vivo with simulating physiological conditions. In a preferred embodiment of this aspect, the invention relates to vital mammalian heart tissue cells maintained ex vivo in a usual culture medium at a physiologically acceptable pH value by applying a culture gas.

Further preferred embodiments of the first aspect of the present invention can be learnt from the subclaims 2 to 8.

In a second aspect, the invention relates to a process for collecting vital mammalian heart tissue cells maintained ex vivo, which process comprises the steps of
collecting heart tissue from the living mammalian heart;
preparing the heart tissue collected in a usual way by cooling in a per se usual preparation medium;
two-dimensional cutting of the heart tissue pieces while obtaining coherent heart tissue cuts;
dividing the heart tissue cuts by means of a glass pipette while obtaining heart tissue fine cuts;
selecting equally shaped intact heart tissue fine cuts; and
submerging and optionally leaving the heart tissue fine cut(s) into/in a suitable usual culture medium while applying a culture gas at a physiologically acceptable pH value.

An alternative embodiment of the invention according to this second aspect relates to a process for collecting vital mammalian heart tissue cells maintained ex vivo, which process comprises the steps of
preparing heart tissue previously collected from heart tissue of a living mammalian heart in a usual way by cooling in a per se usual preparation medium;
two-dimensional cutting of the heart tissue pieces while obtaining coherent heart tissue cuts;
dividing the heart tissue cuts by means of a glass pipette while obtaining heart tissue fine cuts;
selecting equally shaped intact heart tissue fine cuts; and
submerging and optionally leaving the heart tissue fine cut(s) into/in a suitable usual culture medium while applying a culture gas at a physiologically acceptable pH value.

Preferred embodiments of the invention according to the second aspect may be learnt from the subclaims 11 to 20.

In a third aspect, the present invention relates to a process for cultivating vital mammalian heart tissue cells collected ex vivo, which process comprises the step of immersing one or more heart tissue fine cut(s) obtained from a living mammalian heart into a suitable usual culture medium having a physiologically acceptable pH value while applying culture gas and optionally leaving said cut(s) therein.

Preferable embodiments of the invention according to the third aspect may be learnt from the subclaims 22 to 30.

Finally, the invention, in a fourth aspect, relates to the use of said vital mammalian heart tissue cells maintained ex vivo and described above in model investigations of chemical, biological and/or physical influences on physiological or pathophysiological processes.

Preferred embodiments of the invention according to the fourth aspect may be learnt from subclaims 32 to 43.

The heart tissue cells according to the invention are heart tissue cells originating from mammals. Mammals (mammalia) as defined for the purposes of the present invention are any mammals conceivable, as, for example guinea-pigs, mice, dogs, cats or monkeys, and they are humans in preferred embodiments of the invention. Heart tissue cells from humans are preferred, since they provide the best results for the vital tissue model for all conceivable uses as subsequently described in detail.

The mammalian heart tissue cells according to the invention may be maintained ex vivo, i. e. are no longer in a connection with the living mammalian heart. Nevertheless, the cells are vital, i. e. show all functions of the living tissue unit. Particularly, said mammalian heart tissue cells behave like cells in the intact cell unit in the living organ (myocard) so that functional investigations of the interaction between the cells in said unit become possible.

In accordance with the invention, the vital mammalian heart tissue cells are maintained while simulating physiological conditions. According to the invention, “physiological conditions” are understood to define—in the broadest sense—such conditions which are required by mammalian heart tissue cells in order to maintain their vital functions. In accordance with the invention, such conditions are applicable which simulate physiological conditions with respect to aggregate state, temperature, composition, pH value etc. of the medium to such a far-reaching extent that the vital functions of the mammalian heart tissue cells remain maintained. Particularly preferred are conditions in a liquid environment, i. e. conditions of maintaining the mammalian heart tissue cells according to the invention in a suitable liquid culture medium. This has the advantage that the conditions may be established and maintained easily and reproducibly and that the medium may react easily and flexibly to changing requirements with respect to its composition (solutes, pH value, gases) and its physical conditions (temperature, viscosity).

In a preferred embodiment of the invention, the mammalian heart tissue cells are maintained in a usual culture medium. The culture medium may be any culture medium a skilled person knows for storing the cells, maintaining the function of the cells and cultivating the cells. Mixtures of different culture media may be used, too. Advantageously, culture media composed substantially on an aqueous basis are used, but the invention is not restricted to those aqueous media. In addition to water, other liquid components or solvents may be used, too. One or more additive(s) may be added to the culture medium used in accordance with the invention or to the mixture used, which additive(s) allow(s) and/or supports a cultivation of the cells of the invention. Examples of the culture media which may be used in the invention singly or in combination are selected from the group consisting of Opti-MEM I serum-reduced medium, Iscove's modified Dulbecco's medium (IMDM), and Dulbecco's modified Eagle's medium (D-MEM). The latter-mentioned medium is particularly preferred in accordance with the present invention and is a culture medium wherein the culturing step of the vital mammalian heart tissue cells maintained ex vivo in accordance with the present invention is particularly successful. Usual additives are selected from the group of growth factors (e. g. insulin, transferrin), pyruvate, antioxidants (e. g. ascorbic acid, vitamin A, vitamin E, glutathione, lipoic acid), fetal calf serum, amino acids, buffers for adjusting a physiologically acceptable pH value, and other usual additives as, for example, solvents and diluents. A particularly preferred culture medium according to the invention was found to be Dulbecco's modified Eagle's medium (D-MEM)+20% fetal calf serum+2 mM L-glutamine+1% non-essential amino acids (pH 7.4). The components of this culture medium are commercially available from the company Gibco Invitrogen. The mixture indicated by “non-essential amino acids” is a mixture of the amino acids L-alanine, L-asparagine, L-aspartic acid, L-glutamic acid, glycine, L-proline, and L-serine in the amounts 890 mg/l, 1320 mg/l, 1330 mg/l, 1470 mg/l, 750 mg/l, 1150 mg/l and 1050 mg/l, respectively, which mixture is commercially available from the company Gibco Invitrogen with the above name.

A culture gas is added to the culture medium wherein the vital mammalian heart tissue cells maintained ex vivo in accordance with the invention are maintained. The culture gas is a gas which serves the cultivation of the cells and which is dissolved in the culture medium and/or which flows through the culture medium after it was introduced into the culture medium. It is also conceivable that a chemical substance chemically reacting to the culture gas or releasing the culture gas under culture conditions is added into the culture medium. For economical reasons, a culture gas is introduced into the culture medium regularly, whereafter the gas is dissolved at least partially in the liquid medium; the latter step, of course, is dependent upon the culture conditions (temperature, pH value, solvent, etc.). Preferably air is used as the culture gas. In order to avoid the introduction of components impairing the culturing step, the air used must meet advanced requirements. Particularly preferably, clean air or sterile air is used which may be provided or prepared in ways known to a skilled person.

An essential feature of the culture medium is the pH value which has to be adjusted to the physiologically acceptable range. In accordance with the invention, this means that the pH value has to be in a range which does not impair, but which promotes the cultivation of the mammalian heart tissue cells of the invention. In accordance with the invention, the physiological acceptable pH value preferably is a pH value in the range of 6.5 to 8, more preferably a pH value in the range of 6.9 to 7.6, more preferably in the range of 7.3 to 7.5, even more preferably a pH value of 7.4, for example.

The adjustment of the pH value may be carried out in a manner per se known to a skilled person, for example by an addition of chemical substances adjusting the pH value to the physiologically acceptable range. Preferred is the adjustment of the pH value by means of a physiologically acceptable buffer system, which system has the additional advantage that the pH value, once adjusted, remains constant due to the presence of the buffer, if the pH value is shifted due to chemical reactions. Suitable buffers are TRIS (Tris(hydroxymethyl-)amino methane), HEPES (N-2-hydroxyethyl piperazine-N′-ethane sulfonic acid), phosphate buffer, citrate buffer or carbonate buffer. In accordance with the invention, it proved to be preferred to buffer the liquid culture medium by an application of the culture gas (for example clean air) containing CO₂, preferably by an application of clean air having an enhanced content of CO₂, compared to the natural CO₂ content. Particularly preferable, the culture gas contains clean air having a content of 1 to 5% by volume of CO₂, even more preferred clean air having a content of 2 to 3.5% by volume of CO₂ and most preferred clean air having a content of CO₂ of 3.3% by volume. All above % by volume values relate to a temperature of the cultivation step or of the step of feeding the culture gas in a range of the optimum temperature for the cultivation of the cells according to the invention, for example a temperature within the range of 32 to 40° C., preferably a temperature in the range of 34 to 38° C., for example a temperature of 36° C.

In order to feed the inventive vital mammalian heart tissue cells maintained ex vivo with the substances contained in the culture medium and with the culture gas in an optimum way, it is a particularly advantageous and, hence, preferred embodiment of the invention that the vital mammalian heart tissue cells maintained ex vivo are present on a membrane permeable for the culture medium and/or for the culture gas. Said membrane may be designed—in a manner known per se to a skilled person—in such a way that commercially available cell and tissue culture inserts which are arranged in groups on culture plates are provided with suitable natural membranes or synthetic membranes or natural, but synthetically suitably modified membranes on which the inventive vital mammalian heart tissue cells maintained ex vivo are arranged, for example by applying them in the course of the process of collecting and/or cultivating them in accordance with the invention. Suitable membranes are known to a person skilled in this technical field. For example, synthetic membranes having the commercial name Anopore™ (commercialized by the company Nalge Nunc International) may be used, which membranes have a diameter of 25 mm and a pore size of 0.02 μm. The vital mammalian heart tissue cells maintained ex vivo in accordance with the present invention on such natural or synthetic membranes have an optimum exchange of substances with the culture medium and the culture gas so that they are fed continuously with all components necessary for the cultivation. The result are surprisingly long time periods during which an ex vivo cultivation of the cells may be performed without that their natural functions are lost. The latter fact is an optimum precondition for a successful investigation of the functions of the cells according to the invention when using them in the way disclosed by the invention.

In accordance with the invention, it is particularly surprising that mammalian heart tissue cells, specifically human heart tissue cells can be obtained, collected and cultivated while maintaining all vital functions, particularly all heart-specific functions of the cells. When examining the cultivated cells after their withdrawal from the living mammalian tissue and cultivation thereof in accordance with the process described below for 6 days, there could be demonstrated a sufficient vitality of the predominant majority of the cells with the maintenance of the typical cell composition and cell architecture.

The process for collecting vital mammalian heart tissue cells maintained ex vivo in accordance with the present invention comprises as the first step a collection of heart tissue from a living mammalian heart. In a preferred embodiment, heart tissue is obtained and collected from a living human heart. This may be accomplished, for example, at the occasion of surgery actions on the heart (preferably on the human heart), where a connection of the body to a heart-lung machine is required; but the invention is not restricted to this embodiment. For example, in the course of such a surgery action, parts of the so-called atrial auricle are withdrawn. However, mammalian heart tissue may be withdrawn from the living mammalian heart also in other ways which are known to a person skilled in this field.

The heart tissue obtained as described above is prepared in a usual way by cooling in a per se usual preparation medium. This is performed in a way known to a skilled person, for example by a far-reaching removal of fat tissue and connective tissue from the mammalian heart tissue piece obtained from the living heart while cooling. Hence, finally, heart muscle fiber tissue is exposed by the preparation step, preferably. The temperature is in a low-temperature range allowing the preparation to be performed, but also slowing down physiological reactions, preferably within a range of from 0° C. to 6° C., more preferably within a range of from 1° C. to 5° C., even more preferred within a range of from 3 to 4° C.

The preparation medium used in the course of transfer and preparation of the tissue pieces for cooling and storing is a usual preparation medium known to a skilled person for a use in the course of the above steps. For logical reasons, the medium has a pH value in the physiologically acceptable range, preferably in a range of from 6.5 to 8, more preferably in a range of from 7.0 to 7.6, even more preferred in a range of from 7.2 to 7.4, for example of 7.35. In an even more preferred embodiment, the medium contains additional substances advantageous for the maintenance of the tissue pieces, for example growth factors, insulin, transferrin, sodium pyruvate, B-27-supplement (Gibco Invitrogen), folic acid, vitamins, sera (horse, calf, etc.), carbohydrates, specifically sugars (for example glucose, fructose, sucrose, trehalose, etc.), biotin, one or more amino acid(s), for example L-glutamine, one or more buffer(s), for example HEPES or TRIS, and, optionally, also gaseous components or components capable of releasing a gas or gases at the conditions of the preparation step. Particularly advantageously, a medium having the composition of MEM-Hank's medium including 25 mM HEPES, 2 mM L-glutamine (pH 7.35; saturated at 6° C. with O₂) is used as the preparation medium.

The subsequent process step comprises a so-called two-dimensional cutting of the heart tissue pieces obtained in the previous process step while obtaining coherent heart tissue cuts or slices. In a preferred embodiment of the process of the present invention, this step is conducted in two steps. Even more preferred is a two-step cutting process is preferred even more, which carries out the two-dimensional cutting step of the heart tissue pieces in such a way that a first cutting step cuts the heart tissue pieces perpendicularly to the muscle fiber direction at a cutting depth of 0.3 to 3 mm, preferably of 0.8 to 1.2 mm, and a second cutting step cuts the heart tissue pieces parallel to the muscle fiber direction at a cutting depth of 100 to 200 μm. As a result, coherent mammalian heart tissue cuts or slices may be obtained which may be maintained ex vivo and exhibit all properties of living heart tissue. The step of two-dimensional cutting may be performed by means of any apparatus which are available to a skilled person for such purposes. In accordance with the invention, an apparatus is preferably used having the designation McIllwain Chopper and being available from the company The Mickle Laboratory Engineering Co., Guildford, Surrey, United Kingdom. The partly coherent mammalian heart tissue cuts or slices obtained in this process step are separated from each other subsequently in a per se known manner. For this purpose, equipment familiar to a skilled person may be used, for example glass pipettes, lancets or similar equipment. The mammalian heart tissue cuts or slices—as also the mammalian heart tissue fine cuts obtained and separated from each other—are held at the above-mentioned low temperature, preferably in a preparation medium, more preferred in a cold preparation medium, particularly preferred in the above-described preparation medium which is cooled.

Among the mammalian heart tissue fine cuts or slices obtained as described above, those are selected which are uniform and intact as a result of the process of their preparation, i. e. are vital and exhibit the functions known from the living heart. Tests for determining the vitality are described below in the examples.

In accordance with the process of the invention, the selected intact mammalian heart tissue fine cuts or slices are put or immersed into a usual culture medium individually or as several pieces, preferably individually, and are optionally left in said medium, if required for an extended period of time. The culture medium is at a physiologically acceptable pH value, and a culture gas is added thereto.

In a preferred embodiment of the process, one or more of the cut and divided mammalian heart tissue cuts or slices is/are arranged on a culture membrane permeable for the culture medium and for the culture gas before or during the step of immersing it/them into the culture medium. The membrane may be designed—in a manner per se known to a skilled person—in such a way that commercially available cell and tissue culture inserts, which are arranged on culture plates in groups, are provided with suitable natural membranes or synthetic membranes or natural membranes, but modified synthetically in a suitable way. One or more of the vital mammalian heart tissue cells maintained ex vivo in accordance with the invention are arranged on that/those membrane(s), for example by applying the cell(s) thereon before the last step of the collection process according to the invention. Suitable membranes are known to a skilled person. For example, synthetic membranes having the commercial designation Anopore™ (obtained from the company Nalge Nunc International) may be used, which have a diameter of 25 mm and a pore size of 0.02 μm. Mammalian heart tissue cells maintained ex vivo in accordance with the invention on such natural or synthetic membranes exchange solutes and gaseous components with the culture medium and with the culture gas in an optimum manner so that they can be fed with the components necessary for their cultivation in a continuous manner.

As the culture medium, any culture medium may be used which a skilled person knows for the storage of cells, maintenance of the functions of cells and cultivation of cells. Mixtures of different culture media may also be used. Advantageously, culture media are used which are substantially based on an aqueous composition. However, the invention is not restricted to such water-based culture media. In addition to water, there may be contained other liquid components or solvents, too. One or more additive(s) may be added to the culture medium used or to the mixture used in the process of the present invention, which additive(s) allow(s) and or support(s) a storage and/or cultivation of the cells of the invention. Examples of culture media which may be used in accordance with the invention individually or in combination are selected from the group consisting of Opti-MEM I serum-reduced medium, Iscove's modified Dulbecco's medium (IMDM) and Dulbecco's modified Eagle's medium (D-MEM). The latter-mentioned medium is particularly preferred in accordance with the invention and is a culture medium wherein the cultivation and maintenance/storage of the vital mammalian heart tissue cells maintained ex vivo in accordance with the invention is particularly successful. Usual additives are selected from growth factors, insulin, transferrin, sodium pyruvate, B-27-supplement (Gibco Invitrogen), folic acid, vitamins, sera (horse, calf, etc., for example fetal calf serum), carbohydrates, specifically sugars (for example glucose, fructose, sucrose, trehalose, etc.), biotin, amino acids, buffers for adjusting a physiologically acceptable pH value and other usual additives as, for example, solvents and diluents. A particularly preferred culture medium proved to be Dulbecco's modified Eagle's medium (D-MEM)+20% fetal calf serum+2 mM L-glutamine+1% non-essential amino acids (pH 7.4). The components of this culture medium are commercially available from the company Gibco Invitrogen.

In accordance with the present invention, it is also preferred that, as the physiologically acceptable pH value of the culture medium, a pH value in the range of from 6.5 to 8 is adjusted, preferably a pH value in the range of from 6.9 to 7.6, more preferable a pH value in the range of from 7.3 to 7.5, even more preferred a pH value of 7.4. The adjustment of the pH value is performed in the way described above in detail.

In accordance with the invention, the feed of gas to the culture medium may be performed with any conceivable gas maintaining the vitality of the mammalian heart tissue. cells obtained in accordance with the invention. Preferably, air is used as the culture gas, which air—in more preferred embodiments—is clean air or even sterile air. In an even more preferred embodiment of the process, clean air having a CO₂ content above the natural CO₂ content is used as the culture gas, more preferred clean air having a CO₂ content of from 1 to 5% by volume, even more preferred having a CO₂ content of from 2 to 3.5% by volume, for example clean air having a CO₂ content of 3.3% by volume. Without wanting to be bound by this explanation, it is assumed that, in the particularly preferred embodiment of a use of a culture gas enriched with CO₂, the CO₂ enters into a solution equilibrium with the culture medium and contributes to an improved buffering of the afore-mentioned culture medium in the form of the H₂CO₃/HCO₃⁻ buffer system.

The present invention also relates to a process for cultivating vital mammalian heart tissue cells obtained ex vivo. The process of cultivation comprises the step that one or more heart tissue fine cut(s) or slice(s) obtained from the living mammalian heart is/are immersed into a suitable usual culture medium having a physiologically accpetable pH value by adding a culture gas to the medium and, optionally, leaving the cut(s)/slice(s) therein.

In accordance with preferred embodiments of the cultivation process of the invention, the mammalian heart tissue fine cut(s) or slice(s) may be applied to a membrane permeable for the culture medium and the culture gas before the step of immersing it/them into the culture medium. One or more mammalian heart tissue fine cut(s) or slice(s) may be applied per membrane. For the selection of the membrane and its origin, the same criteria may be applied in further preferred embodiments of the cultivation process of the invention as they were described above in connection with the process for collecting the mammalian heart tissue cells of the invention. The same is applicable to the culture media useable in the invention as well as to their preferred composition: They were also described above in detail in connection to the last step of the process for collecting the mammalian heart tissue cells, and the above description of the general and preferred embodiments is applicable, mutatis mutandis, to the present cultivation process.

In further preferred embodiments, as the physiologically acceptable pH value, a pH value may be adjusted in the present cultivation process, which is in the range of from 6.5 to 8, preferably a pH value in the range of from 6.9 to 7.6, more preferably a pH value in the range of from 7.3 to 7.5, for example a pH value of 7.4.

The feed of culture gas may be a feed of any suitable gas supporting the cultivation of the mammalian heart tissue cells. Further preferred is a cultivation process wherein the feed of culture gas is a feed of clean air, more preferred a feed of clean air having an increased content of CO₂, compared to the natural CO₂ content, even more preferred clean air having a CO₂ content of from 1 to 5% by volume, mostly preferred clean air having a CO₂ content of from 2 to 3.5% by volume, for example clean air having a CO₂ content of 3.3% by volume. The temperature of the culture medium in said cultivation process is preferably 34 to 38° C., more preferred 36 to 37° C.

In the frame of the above-described process for collecting the mammalian heart tissue cells according to the invention, and in the frame of the latter-described process for cultivating such mammalian heart tissue cells, mammalian heart tissue cells, which may be maintained ex vivo, are obtained while maintaining their vital functions. The time periods of a complete maintenance of all specific functions of such cells vary with the living starting material, with the conditions of their collection and with the culture media and culture gases used. In preferred embodiments, these time periods are up to 7 days or more, as may be learnt from the examples.

Further preferred embodiments of the processes of the present invention comprise the common cultivation of vital mammalian heart tissue cells maintained ex vivo according to the invention and of cells and/or tissue pieces of a different origin. In this respect, the simultaneous cultivation of different cells is particularly preferred. The cells and/or tissue pieces of a different origin cultivated together with the mammalian heart tissue cells of the invention may be any conceivable cells or tissue pieces, respectively. Particularly preferred are other mammalian cells and/or mammalian tissue pieces, more preferably other human cells and/or human tissue pieces, than heart tissue cells or heart tissue pieces. Foreign body cells and/or foreign body tissue pieces are also useable for the common—preferably common simultaneous—cultivation with the mammalian heart tissue cells according to the invention. Preferred foreign body cells are bacterial cells, viral cells and/or fungal cells; however, the invention is not restricted to those cells in these embodiments.

The invention also relates to a use of the vital mammalian heart tissue cells maintained ex vivo in accordance with the invention as described above, in particular the use of the mammalian heart tissue cells obtained in accordance with the process of the invention, in model investigations of chemical, biological and/or physical influences on physiological or pathophysiological processes, particularly preferred on such processes of the mammalian heart and even more preferred on such processes of the human heart.

By providing the mammalian heart tissue cells, methodical functional investigations of the human and animal atrial myocard become possible for the first time. Inter alia, the effects of receptor agonists and antagonists on the myocardial signal transduction may be investigated by using molecular biological and electro-physiological methods.

The tissue cuts or slices, respectively, maintained in a culture may also be stimulated by means of carbon fiber electrodes via an “electrical field stimulation”. By such a stimulation and by varying the voltage applied, the electrodes and the stimulus configuration, respectively, the frequency of the pulses applied and of the rhythm thereof, respectively, arrhythmiae of the heart may be simulated. The effects on the tissue may be analyzed subsequently by pathological investigation methods (histology, immuno-histochemistry etc.), electrophysiological investigation methods (microelectrode techniques, patchclamp etc.) and molecular investigation methods (Western Blotting, PCR etc.).

For the atrial tissue, in this connection the investigation during or after a rapid electric stimulation appears to be of particular importance, since the changes of the atrial tissue of patients having tachycardial atrial arrhythmiae (particularly having atrial fibrillation) may be reproduced by means of this type of stimulation. Since the atrial walls are very thin, a sequential biopsy of the atria cannot be performed in vivo. Hence, the model of cultivating atrial tissue provided by the present invention, accompanied by an electric stimulation thereof, allows functional analyses of the intact tissue unit to be performed, for the first time.

The investigations according to the invention showed that a rapid electric stimulation of atrial tissue results into the same cellular changes of the gene expression within 24 hours as they can be detected in patients suffering from atrial fibrillation. There may be detected an enhanced expression of the “angiotensin-converting enzyme” (ACE), a regulation of the angiotensin II receptors as well as an activation of the “mitogen-activated protein kinase” (MAP-kinase Erk2) (see the following example 2), as they could be detected earlier in patients suffering from atrial fibrillation.

In a manner analogous to the atrial fibrillation, effects of tachycardial arrhythmiae in the ventricular tissue may be investigated similarly. In this respect, investigations of the influence of large electric fields (simulation of a defibrillation or cardioversion) on the myocardium are of great interest.

An electrical stimulation of tissue units cultivated ex vivo is conducted, for example, by means of carbon electrodes via electric field stimulation. The carbon electrodes are connected to a commercially available stimulator via platinum wires. For the stimulation, monophasic or biphasic direct current stimuli of about 150 V are applied in the medium, and the mammalian heart tissue cells maintained in the culture are positioned in the center of the electrical field. The frequency of the stimulation and the duration of the stimulus may be varied in accordance with the electrophysiological requirements in order to guarantee a sufficient electrical stimulation of the tissue. The answer of the tissue to the stimulus may be controlled by means of microelectrode systems or by means of direct light microscopical observation. For a long term stimulation, the stimulating electrodes are connected to the cell culture box via extension cords so that a stimulation of several days' duration under optimum temperature conditions can be realized.

In particular, the vital mammalian heart tissue cells maintained ex vivo in accordance with the invention may be used in the following areas:
Defined exogenous stimuli, which comprise chemical, biological and physical stimuli, act on the heart tissue, and the effects of those stimuli on the morphology and function of the heart tissue and its components are determined.
The mammalian heart tissue cells are used for the target identification and target validation, for the identification and validation of diagnostic markers and for the development of diagnostic tools for an early recognition or acute diagnosis, respectively, of cardiovascular diseases.
The mammalian heart tissue cells are used for the elucidation of physiological, preferably pathophysiological mechanisms of cardiovascular diseases, preferably of cardiac arrhythmiae, of ischemic diseases and in the elucidation of preconditioning effects for the development of drugs.
The mammalian heart tissue cells are used for a screening and an identification of effective substances and for the validation including the use as a toxicity assay.
The mammalian heart tissue cells are used for the development of drugs for the treatment of diseases of the cardiovascular system.
Chemical stimuli are produced by biologically or pharmacologically effective substances or by substances which serve for testing and developing preventive or therapeutically relevant substances.
(Micro-) Biological stimuli, which may influence cellular functions, are produced by bacteria, viruses, fungi, unicellular organisms or their components, respectively, as, for example, haptens, antibodies or antigens having human or animal origin, peptides, proteins, DNA, RNA or other macromolecules.
Physical stimuli are produced by electromagnetic or radioactive radiation, electrical stimulation, mechanical stimuli (preferably tension), changes of temperature, of pressure or of oxygen content or carbon dioxide content of the air or of the culture medium.
Function(s) of the mammalian heart tissue, preferably of the human heart tissue, is/are their cellular vitality, their tissue-specific gene expression on the mRNA level and protein level, their ionic homeostasis, their metabolism, their signal transduction, their capability of regeneration and division in cases of cells having said capability, their capability to be stimulated by electric stimuli, their electric conductivity and/or their contractility.
The morphology of the heart tissue is/are the number, relative frequency, localization, arrangement, shape and/or size of all cells and cell types present in the tissue, preferably of the monocytes, fibroblasts, leucocytes, nerve cells and endothelial cells.
The morphology of the heart tissue is/are the subcellular characteristics of the cell types, preferably the number and size of mitochondria, other cell organelles, and/or the integrity of the contractile structure or of the cytoskeleton.

The invention is further explained in detail by the subsequent examples without being restricted to those examples.

### EXAMPLE 1

### Collection and Investigation of the Vitality of Human Heart Tissue Cells (Atrial Cuts)

For the preparation of the tissue cuts or slices, human heart tissue was used. The tissue consisted of parts of the atrial auricle.

The tissue was removed in the course of a surgery action on the heart, during which the connection to a heart-lung machine was necessary. The tissue slices were transferred and prepared while cooling with ice to a temperature of 4° C. in a preparation medium having the following composition: MEM-Hank's medium+25 mM HEPES+2 mM L-glutamine (pH 7.35 at 6° C., saturated with O₂).

Fat tissue and connecting tissue were largely removed from the tissue piece withdrawn; hence, mainly muscle fiber tissue remained. The tissue slices were obtained by two-dimensional cutting by means of a McIllwain Chopper (The Mickle Laboratory Engineering Co., Guildford, Surrey, U.K.). As the first step, cutting was performed perpendicular to the fiber direction with a cutting depth of 1 mm, and as the second step, the resulting fragments were divided parallel to the fiber direction with a cutting depth of 150 μm.

In an ice-cooled preparation buffer, the separation of still coherent tissue pieces was performed by means of a glass pipette.

For the cultivation step, intact and uniform tissue cuts/slices were selected. They were transferred onto Anapore™ membranes (25 mm, pore size 0.02 μm) of cell and tissue cultivation inserts (obtained from the company Nunc, Wiesbaden, Germany). As a rule, about 5 to 10 slices were cultivated on one membrane. After the application of the tissue slices, the inserts were inserted into Nunclon™ cell and tissue culture 6-well plates (obtained from the company Nunc, Wiesbaden, Germany). The plates contained 1.2 ml culture medium per well. The culture medium had the following composition: Dulbecco's modified Eagle's medium (D-MEM)+20% fetal calf serum+2 mM L-glutamine+1% non-essential amino acids (pH 7.4) (all substances were obtained from the company Gibco Invitrogen). In the course of the cultivation, the cultures were fed with gas, i. e. 3.3% CO₂, at 36° C. The media were changed three times per week.

After 6 days of cultivation, investigations of the vitality of the cultivated explantates were carried out.

The cells were coloured with propidium iodide (red colour) for showing dead cell nuclei as well as with SYTO13 (green colour) (obtained from the company Molecular Probes, Eugene, Oreg., U.S.A.) for showing vital cell nuclei, as was described by Lendeckel et al., J. of Ethnopharmacology 79 (2002), 221-227.

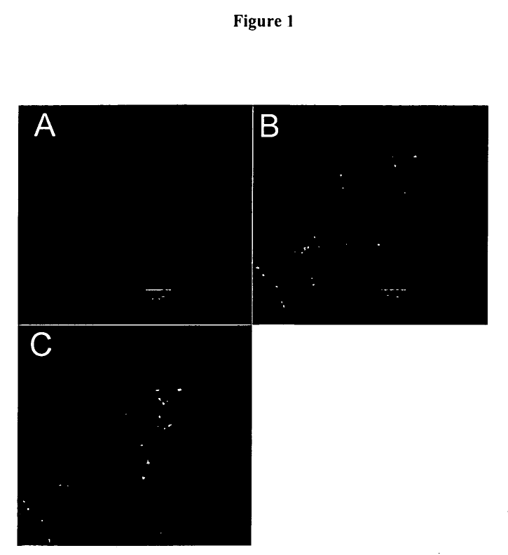

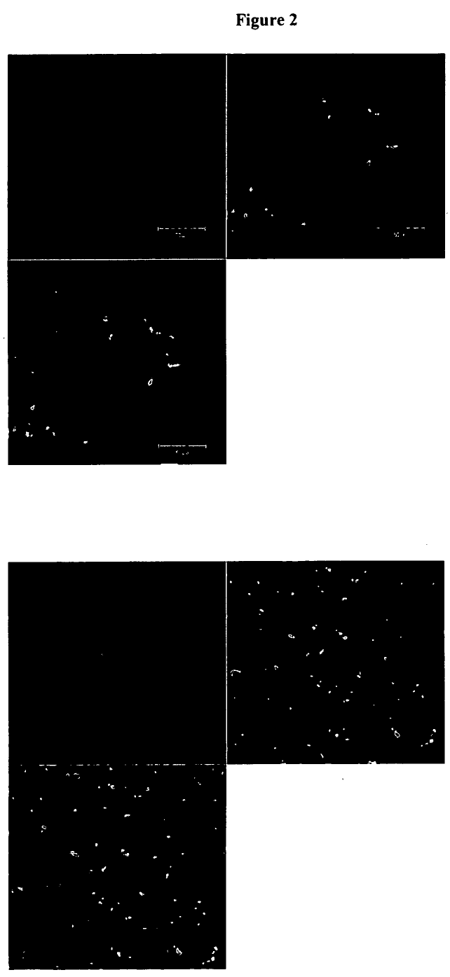

The results are shown in FIGS. 1 and 2. FIG. 1A shows the red fluorescence; FIG. 1B shows the green fluorescence; and FIG. 1C shows a superposition of FIGS. 1A and 1B. FIG. 2 shows the results of investigations of the vitality of human atrial slices after 8 days of cultivation in vitro. The coloration is the same as described in connection with FIG. 1. However, FIG. 2 shows a better magnification. FIGS. 2A and 2D show the coloration with propidium iodide; FIGS. 2B and 2E show the coloration with SYTO13; and FIGS. 2C and 2F show the superposition of the FIGS. 2A+2B and of the FIGS. 2D+2E.

FIGS. 1 and 2 show that the predominant majority of the cells is vital after a cultivation in vitro of human atrial slices for several days and that the typical cell composition and tissue architecture is retained.

### EXAMPLE 2

### Induction of the Expression of the “Angiotensin-Converting Enzyme” (ACE) as Well as of the MAP-Kinase Erk2 in In Vitro Electrically Stimulated Slices of Atrial Tissue

The tissue slices obtained in accordance with Example 1 were electrically stimulated for a time period of 24 hours or were cultivated without an electrical stimulation under identical conditions (with the exception of the stimulation). The electrical stimulation was performed with a voltage of 12 V/cm, and the frequency was 36 min⁻¹ (corresponding to a sinus rhythm) or 120 min⁻¹ (corresponding to atrial fibrillation). When simulating the atrial fibrillation (frequency of 120 min⁻¹), a strong induction of the expression of the ACE mRNA and Erk2 mRNA was observed, compared to the non-stimulated controls. These changes are not observed at the low-frequency stimulation (sinus rhythm, SR). Quite to the contrary: Under these conditions, the ACE expression and Erk2 expression are reduced. The same effects relating to the expression of ACE and Erk2 were already observed in ex vivo material of patients suffering from atrial fibrillation in comparison to patients with SR [Goette, A. et al., Regulation of angiotensin II receptor subtypes during atrial fibrillation in humans; Circulation 101 (2000), 2678-2681; Goette, A. et al., Increased expression of extracellular signal-regulated kinase and angiotensin-converting enzyme in human atria during atrial fibrillation; Journal of the American College of Cardiology 35:1669-1677, 2000].

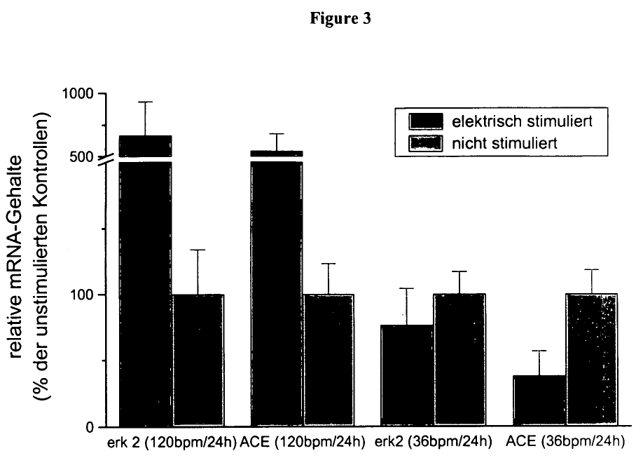

FIG. 3 shows the mRNA expression of ACE and Erk2 in heart tissue slices after a cultivation ex vivo.

### EXAMPLE 3

### Induction of the Expression of the κ1-Opiate Receptor (KOR) at the mRNA Level after an Electrical Stimulation of In Vitro-Differentiated Cardiomyocytes (P19 Cells) as Well as in Human Atrial Tissue Slices

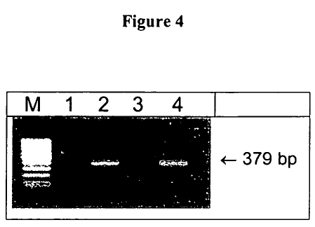

From cells or tissues prepared in accordance with example 1, the whole RNA was isolated by standard methods after 24 hours of electrical stimulation. The detection of KOR-mRNA was performed by means of the Polymerase Chain Reaction (PCR) and presentation of the PCR products by means of agarose gel electrophoresis and coloration by ethidium bromide. FIG. 4 shows the evidence of KOR-mRNA in electrically stimulated in vitro-differentiated cardiomyocytes (P19 cells) as well as in electrically stimulated human atrial slices. A KOR expression was observed in P19 cells (tracks 1 and 2) and in tissue slices as well (tracks 3 and 4), but exclusively after a previous electrical stimulation (120 bpm) (tracks 2 and 4).

1. Vital mammalian heart tissue cells maintained ex vivo while simulating physiological conditions.

2. The mammalian heart tissue cells maintained ex vivo according to claim 1 in a usual culture medium with the addition of a culture gas at a physiologically acceptable pH value.

3. The mammalian heart tissue cells according to claim 2, wherein the physiologically acceptable pH value is a pH value in the range of from 6.5 to 8, preferably a pH value in the range of from 6.9 to 7.6, more preferably a pH value in the range of from 7.3 to 7.5, even more preferable a pH value of 7.4.

4. The mammalian heart tissue cells according to claim 2, wherein the culture medium is Opti-MEM I serum-reduced medium, Iscove's modified Dulbecco's medium (IMDM) or Dulbecco's modified Eagle's medium (D-MEM), preferably Opti-MEM I serum-reduced medium+2 mM L-glutamine+1% non-essential amino acids+B27 supplement or Dulbecco's modified Eagle's medium+20% fetal calf serum+2 mM L-glutamine+1% non-essential amino acids (pH 7.4).

5. The mammalian heart tissue cells according to claim 2, wherein the culture gas is clean air, preferably clean air having an enhanced content of CO₂, more preferred clean air containing 1 to 5% by volume of CO₂, even more preferred clean air containing 2 to 3.5% by volume of CO₂.

6. The mammalian heart tissue cells according to claim 2 on a membrane permeable for the culture medium and/or for the culture gas.

7. The mammalian heart tissue cells according to claim 1 from the human heart.

8. The mammalian heart tissue cells according to claim 1 having maintained all vital functions, preferably all heart-specific functions, of the cells.

9. A process for collecting vital mammalian heart tissue cells maintained ex vivo, said process comprising the steps of
obtaining heart tissue from the living mammalian heart;
preparing the heart tissue obtained in a usual way while cooling in a per se usual preparation medium;
two-dimensional cutting of the heart tissue pieces while obtaining coherent heart tissue cuts;
dividing the heart tissue cuts by means of a glass pipette so as to obtain heart tissue fine cuts;
selecting uniform intact heart tissue fine cuts; and
immersing, and optionally leaving, the heart tissue fine cut(s) into/in a suitable usual culture medium while feeding culture gas at a physiologically acceptable pH value.

10. A process for collecting vital mammalian heart tissue cells maintained ex vivo, said process comprising the steps of
preparing heart tissue previously obtained from heart tissue from a living mammalian heart on a usual route while cooling in a per se usual preparation medium;
two-dimensional cutting of the heart tissue pieces while obtaining coherent heart tissue cuts;
dividing the heart tissue cuts by means of a glass pipette so as to obtain heart tissue fine cuts;
selecting uniform intact heart tissue fine cuts; and
immersing, and optionally leaving, the heart tissue fine cut(s) into/in a suitable usual culture medium while feeding culture gas at a physiologically acceptable pH value.

11. The process according to claim 9, wherein human heart tissue is used as the mammalian heart tissue.

12. The process according to claim 9, wherein a usual preparation medium having a pH value in the physiologically acceptable range is used as the preparation medium, preferably wherein MEM-Hank's medium including 25 mM HEPES, 2 mM L-glutamine (pH 7.35; at 6° C. saturated with O₂) is used.

13. The process according to claim 9, wherein heart muscle fiber tissue is used predominantly.

14. The process according to claim 9, wherein a temperature in the range of from 0 to 6° C. is adjusted as the temperature of the cooling step, preferably a temperature in the range of from 1 to 5° C., even more preferably a temperature of from 3 to 4° C.

15. The process according to claim 9, wherein the two-dimensional cutting of the heart tissue slices is performed in two steps.

16. The process according to claim 15, wherein the two-dimensional cutting of the heart tissue pieces is performed in such a manner that, in the first step, cutting is performed perpendicularly to the muscle fiber direction with a cutting depth in the range of from 0.3 to 3 mm, preferably of from 0.8 to 1.2 mm, and in the second step, cutting is performed on the resulting fragments parallel to the muscle fiber direction with a cutting depth in the range of from 100 to 200 μm.

17. The process according to claim 9, wherein the step of dividing the heart tissue pieces is performed with a glass pipette in a preparation medium, preferably in a cold preparation medium, more preferred in a cold preparation medium as claimed in claim 12.

18. The process according to claim 9, wherein the cut and divided heart tissue slices are put on a culture membrane permeable for the culture medium and for the culture gas before or during the step of immersing into the culture medium.

19. The process according to claim 9, wherein a pH value in the range of from 6.5 to 8 is adjusted as the physiologically acceptable pH value, preferably a pH value in the range of from 6.9 to 7.6 is adjusted, more preferably a pH value in the range of from 7.3 to 7.5 is adjusted, even more preferred a pH value of 7.4 is adjusted.

20. The process according to claim 9, wherein a gas feed of clean air is added to the tissue slices, preferably a gas feed of clean air containing 1 to 5% by volume CO₂ is added, more preferably a gas feed of clean air containing 2 to 3.5% by volume CO₂ is added.

21. A process for cultivating vital mammalian heart tissue cells obtained ex vivo, said process comprising the step of immersing one or more heart tissue fine cut(s) collected from a living mammalian heart into a suitable usual culture medium having a physiologically acceptable pH value while feeding a culture gas and, optionally, leaving it/them therein.

22. The process according to claim 21, wherein the heart tissue slice(s) is/are put on a culture membrane permeable for the culture medium and for the culture gas before or during the step of immersing into the culture medium.

23. The process according to claim 21, wherein human heart tissue cells are used as the mammalian heart tissue cells.

24. The process according to claim 21, wherein a pH value in the range of from 6.5 to 8 is adjusted as the physiologically acceptable pH value, preferably a pH value in the range of from 6.9 to 7.6 is adjusted, more preferably a pH value in the range of from 7.3 to 7.5 is adjusted, even more preferred a pH value of 7.4 is adjusted.

25. The process according to claim 21, wherein a gas feed of clean air is added to the tissue slices, preferably a gas feed of clean air containing 1 to 5% by volume CO₂ is added, more preferably a gas feed of clean air containing 2 to 3.5% by volume CO₂ is added.

26. The process according to claim 21, wherein the temperature of the culture medium is 34 to 38° C., preferably is 36 to 37° C.

27. The process according to claim 9, wherein the cultivation is performed in Opti-MEM I serum-reduced medium+2 mM L-glutamine+1% non-essential amino acids+B27 supplement or in Dulbecco's modified Eagle's medium+20% fetal calf serum+2 mM L-glutamine+1% non-essential amino acids (pH 7.4).

28. The process according to claim 9, while maintaining vital, preferably heart-specific, functions of the heart muscle tissue unit.

29. The process according to claim 9, comprising the common, preferably the simultaneous cultivation of mammalian heart tissue cells and cells and/or tissue pieces of a different origin.

30. The process according to claim 29, wherein the cells or tissue pieces of a different origin are mammalian cells and/or mammalian tissue pieces, preferably human cells and/or human tissue pieces, and/or foreign body cells and/or foreign body tissue pieces, preferably cells selected from the group of bacterial cells, viral cells and fungal cells.

31. A method of model investigating utilizing vital mammalian heart tissue cells maintained ex vivo according to claim 1 in model investigations of chemical, biological and/or physical influences on physiological or pathophysiological processes.

32. The method according to claim 31 in model investigations of chemical, biological and/or physical influences on physiological or pathophysiological processes of the mammalian heart, preferably of the human heart.

33. The method of claim 31, wherein defined exogenous stimuli comprising chemical, biological and physical stimuli are produced on the heart tissue and the effects of these stimuli on the morphology and function of the heart tissue and its components are determined.

34. The method of claim 31 for the target identification and target validation, for the identification and validation of diagnostic markers and for the development of diagnostic tools for an early recognition or acute diagnosis of cardiovascular diseases.

35. The method of claim 31 for the elucidation of physiological, preferably pathophysiological mechanisms of cardiovascular diseases, preferably of cardiac arrhythmiae, of ischemic diseases and in the elucidation of preconditioning effects for the development of drugs.

36. The method according to claim 31 for a screening and an identification of effective substances and for the validation including the use as a toxicity assay.

37. The method according to claim 31 for the development of drugs for the treatment of diseases of the cardiovascular system.

38. The method according to claim 31, wherein chemical stimuli are produced by biologically or pharmacologically effective substances or by substances which serve for testing and developing preventive or therapeutically relevant substances.

39. The method according to claim 31, wherein (micro-) biological stimuli, which may influence cellular functions, are produced by bacteria, viruses, fungi, unicellular organisms or their components, respectively, as, for example, haptens, antibodies or antigens having human or animal origin, peptides, proteins, DNA, RNA or other macromolecules.

40. The method according to claim 31, wherein physical stimuli are produced by electromagnetic or radioactive radiation, electrical stimulation, mechanical stimuli (preferably tension), changes of temperature, of pressure or of oxygen content or carbon dioxide content of the air or of the culture medium.

41. The method according to claim 31, wherein function(s) of the mammalian heart tissue, preferably of the human heart tissue, is/are their cellular vitality, their tissue-specific gene expression on the mRNA level and protein level, their ionic homeostasis, their metabolism, their signal transduction, their capability of regeneration and division in cases of cells having said capability, their capability to be stimulated by electric stimuli, their electric conductivity and/or their contractility.

42. The method according to claim 31, wherein the morphology of the heart tissue is/are the number, relative frequency, localization, arrangement, shape and/or size of all cells and cell types present in the tissue, preferably of the monocytes, fibroblasts, leucocytes, nerve cells and endothelial cells.

43. The method according to claim 31, wherein the morphology of the heart tissue is/are the subcellular characteristics of the cell types, preferably the number and size of mitochondria, other cell organelles, and/or the integrity of the contractile structure or of the cytoskeleton.

44. The mammalian heart tissue cells according to claim 2 from the human heart.

45. The mammalian heart tissue cells according to claim 2 having maintained all vital functions, preferably all heart-specific functions, of the cells.

46. The mammalian heart tissue cells according to claim 3 from the human heart.

47. The mammalian heart tissue cells according to claim 3 having maintained all vital functions, preferably all heart-specific functions, of the cells.

48. The mammalian heart tissue cells according to claim 4 from the human heart.

49. The mammalian heart tissue cells according to claim 4 having maintained all vital functions, preferably all heart-specific functions, of the cells.

50. The mammalian heart tissue cells according to claim 5 from the human heart.

51. The mammalian heart tissue cells according to claim 5 having maintained all vital functions, preferably all heart-specific functions, of the cells.

52. The mammalian heart tissue cells according to claim 6 from the human heart.

53. The mammalian heart tissue cells according to claim 6 having maintained all vital functions, preferably all heart-specific functions, of the cells.

54. The mammalian heart tissue cells according to claim 7 having maintained all vital functions, preferably all heart-specific functions, of the cells.

55. The process according to claim 10 wherein human heart tissue is used as the mammalian heart tissue.

56. The process according to claim 10, wherein a usual preparation medium having a pH value in the physiologically acceptable range is used as the preparation medium, preferably wherein MEM-Hank's medium including 25 mM HEPES, 2 mM L-glutamine (pH 7.35; at 6° C. saturated with O₂) is used.

57. The process according to claim 10 wherein heart muscle fiber tissue is used predominantly.

58. The process according to claim 10 wherein a temperature in the range of from 0 to 6° C. is adjusted as the temperature of the cooling step, preferably a temperature in the range of from 1 to 5° C., even more preferably a temperature of from 3 to 4° C.

59. The process according to claim 10 wherein the two-dimensional cutting of the heart tissue slices is performed in two steps.

60. The process according to claim 10 wherein the step of dividing the heart tissue pieces is performed with a glass pipette in a preparation medium, preferably in a cold preparation medium, more preferred in a cold preparation medium as claimed in claim 12.

61. The process according to claim 10 wherein the cut and divided heart tissue slices are put on a culture membrane permeable for the culture medium and for the culture gas before or during the step of immersing into the culture medium.

62. The process according to claim 10 wherein a pH value in the range of from 6.5 to 8 is adjusted as the physiologically acceptable pH value, preferably a pH value in the range of from 6.9 to 7.6 is adjusted, more preferably a pH value in the range of from 7.3 to 7.5 is adjusted, even more preferred a pH value of 7.4 is adjusted.

63. The process according to claim 10 wherein a gas feed of clean air is added to the tissue slices, preferably a gas feed of clean air containing 1 to 5% by volume CO₂ is added.

64. The process according to claim 10 wherein the cultivation is performed in Opti-MEM I serum-reduced medium+2 mM L-glutamine+1% non-essential amino acids+B27 supplement or in Dulbecco's modified Eagle's medium+20% fetal calf serum+2 mM L-glutamine+1% non-essential amino acids (pH 7.4).

65. The process according to claim 10 while maintaining vital, preferably heart-specific, functions of the heart muscle tissue unit.

66. The process according to claim 10 comprising the common, preferably the simultaneous cultivation of mammalian heart tissue cells and cells and/or tissue pieces of a different origin.
